(19) United States
(12) Patent Application Publication
(10) Pub. No.: US 2024/0133886 A1
(43) Pub. Date: Apr. 25, 2024

(54) METHODS OF ASSESSING CELL PRODUCTS

(71) Applicant: ADOC SSF , LLC, South San Francisco, CA (US)

(72) Inventors: Bhamini Purandare, South San Francisco, CA (US); Barbara Sennino, South San Francisco, CA (US)

(73) Assignee: ADOC SSF , LLC, South San Francisco, CA (US)

(21) Appl. No.: 18/535,302

(22) Filed: Dec. 11, 2023

Related U.S. Application Data
(63) Continuation of application No. PCT/US2022/033001, filed on Jun. 10, 2022.
(60) Provisional application No. 63/209,557, filed on Jun. 11, 2021.

(51) Int. Cl. G01N 33/569; G01N 33/551

(52) U.S. Cl. CPC G01N 33/56972; G01N 33/551; G01N 2333/70539; G01N 2333/70596

(57) ABSTRACT

Methods of assessing or evaluating functional properties of T cell therapeutic products are described herein.

### CROSS-REFERENCE TO RELATED APPLICATIONS

This application is a Continuation Application of International Patent Application No. PCT/US2022/033001, filed Jun. 10, 2022, which claims the benefit of priority of U.S. Provisional Application No. 63/209,557, filed Jun. 11, 2021, the content of each of which is incorporated by reference herein in its entirety for any purpose and to each of which priority is claimed.

### FIELD

Methods of assessing or evaluating Cell Products are provided, comprising contacting the Cell Product with a polypeptide, wherein the polypeptide comprises an epitope peptide sequence that forms an epitope peptide-HLA complex, wherein the T cells of the Cell Product express or are engineered to express a certain T cell receptor. In some embodiments, the Cell Products are therapeutic Cell Products for use in treating a disease in a subject.

### BACKGROUND

T cells are the primary mediators of adaptive immunity. Directed by the specificity of each T cell's unique T cell receptor (TCR), T cells regulate autoimmunity, help activate B cells and innate effectors, and directly kill infected and cancerous cells in a precisely targeted manner. Engineered Cell Products, such as NeoTCR, CAR-T cell, and tumor-infiltrating lymphocyte (TIL) products, are an important class of therapy for cancer and many are designed specifically for each patient. Prior to administration to a patient, functional analysis of each Cell Product is important in order to ensure, for example, that the Cell Product is potent and effective. Such pharmacological evaluation of Cell Products, including evaluation of antigen-specific cell killing, effector cytokine secretion, and proliferative activity on contact with cognate antigen-expressing tumor cells is typically performed using target cells. Such assays have limitations such as low reproducibility, being low-throughout, and having the requirement to generate a new target cell line for each product in some cases.

Accordingly, there is a need for assays to evaluate the efficacy, potency, and/or specificity of Cell Products in a quantitative manner that overcomes these limitations.

### SUMMARY

The present inventions described herein provide for methods of assessing or evaluating Cell Products. Accordingly, some embodiments are provided below.

### Embodiment 1

A method of evaluating a Cell Product, comprising contacting the Cell Product with a polypeptide, wherein the polypeptide comprises an epitope peptide sequence, an HLA class I sequence, and a β2-microglobulin sequence, and forms an epitope peptide-HLA complex; wherein the Cell Product comprises a plurality of T cells, wherein at least one T cell of the Cell Product expresses a select T cell receptor (TCR), wherein the select TCR recognizes the epitope peptide-HLA complex; and detecting at least one marker of T cell function, wherein at least one marker of T cell function is selected from a proliferation marker, a cytotoxicity marker, and an activation marker.

### Embodiment 2

The method of embodiment 1, wherein the polypeptide is bound to a solid support.

### Embodiment 3

The method of embodiment 2, wherein the solid support is a well of a multiwell plate, beads, or cells.

### Embodiment 4

The method of embodiment 2 or embodiment 3, wherein the polypeptide comprises or is conjugated to a first member of a binding pair, and the solid support comprises a second member of a binding pair.

### Embodiment 5

The method of embodiment 4, wherein the binding pair is biotin and streptavidin or biotin and avidin.

### Embodiment 6

The method of any one of embodiments 2-5, wherein the solid support is a streptavidin-coated multiwell plate.

### Embodiment 7

The method of any one of embodiments 2-5, wherein the solid support is streptavidin-coated beads.

### Embodiment 8

The method of embodiment 7, wherein the beads have an average diameter of 1-10 μm.

### Embodiment 9

The method of any one of embodiments 2-5, wherein the solid support is cells, and wherein the cells have streptavidin-conjugated antibodies bound to the surface.

### Embodiment 10

The method of any one of embodiments 6-9, wherein the polypeptide is conjugated to biotin.

### Embodiment 11

The method of any one of embodiments 1-10, wherein the proliferation marker is selected from Ki67, cell proliferation dyes, Proliferating Cell Nuclear Antigen (PCNA), and Minichromosome Maintenance Complex Component 2 (MCM2).

### Embodiment 12

The method of embodiment 11, wherein Ki67 is detected using an anti-Ki67 antibody.

### Embodiment 13

The method of embodiment 12, wherein the T cells are permeabilized prior to contact with the anti-Ki67 antibody.

### Embodiment 14

the method of any one of embodiments 1-13, wherein the proliferation marker is detected using flow cytometry.

### Embodiment 15

The method of any one of embodiments 1-14, wherein the cytotoxicity marker is selected from CD107a, perforin, and granzyme B.

### Embodiment 16

The method of embodiment 15, wherein CD107a is detected using an anti-CD107a antibody.

### Embodiment 17

The method of embodiment 15 or embodiment 16, wherein the cytotoxicity marker is detected using flow cytometry.

### Embodiment 18

The method of any one of embodiments 1-17, wherein the activation marker is selected from IFNγ, IL2, TNFα, 4-1BB, OX40, and CD25.

### Embodiment 19

The method of embodiment 18, wherein the activation marker is detecting using a cytometric bead array.

### Embodiment 20

The method of any one of the preceding embodiments, wherein the Cell Product comprises CD8+ and/or CD4+ T cells from a subject.

### Embodiment 21

The method of embodiment 20, wherein the subject has cancer.

### Embodiment 22

The method of any one of the preceding embodiments, wherein the Cell Product is enriched for T cells that express the select TCR.

### Embodiment 23

The method of any one of the preceding embodiments, wherein the Cell Product has been engineered to express the select TCR.

### Embodiment 24

The method of embodiment 23, wherein the select TCR is preset in the T cell genome at the endogenous TCR locus.

### Embodiment 25

The method of embodiment 23 or embodiment 24, wherein the Cell Product is produced by a non-viral engineering method.

### Embodiment 26

The method of any one of embodiments 23-25, wherein the Cell Product is produced using CRISPR.

### Embodiment 27

The method of any one of embodiments 23-26, wherein the Cell Product does not comprise any exogenous DNA sequences in the genome of the T cells.

### Embodiment 28

The method of any one of the preceding embodiments, wherein the select TCR binds a neoepitope comprising an amino acid mutation resulting from a somatic coding mutation present in a cancer.

### Embodiment 29

The method of embodiment 28, wherein the Cell Product comprises CD8+ and/or CD4+ T cells from a subject with cancer, and wherein the neoepitope comprises an amino acid mutation resulting from a somatic coding mutation present in the subject's cancer.

### Embodiment 30

The method of any one of the preceding embodiments, wherein the Cell Product is a therapeutic product.

### Embodiment 31

The method of any one of the preceding embodiments, wherein the method comprises determining the percentage of T cells in the Cell Product that express the select TCR.

### Embodiment 32

The method of any one of the preceding embodiments, wherein the method comprises: contacting a solid support with the polypeptide, wherein the polypeptide comprises a first member of a binding pair and the solid support comprises a second member of a binding pair; removing unbound polypeptide; contacting the solid support-bound polypeptide with the Cell Product under conditions suitable for the select TCR to bind to the epitope peptide-HLA complex; and detecting at least one marker of T cell function.

### Embodiment 33

The method of any one of the preceding embodiments, wherein the method comprises determining whether the Cell Product meets a quality control standard.

### Embodiment 34

The method of any one of the preceding embodiments, wherein the method comprises determining the appropriate dose of the Cell Product to administer to a subject.

### Embodiment 35

The method of any one of the preceding embodiments, wherein the method further comprises administering the Cell Product to a subject.

### Embodiment 36

The method of embodiment 34 or embodiment 35, wherein the subject has cancer.

### Embodiment 37

The method of embodiment 36, wherein the cancer is selected from melanoma, lung cancer, breast cancer, head and neck cancer, ovarian cancer, prostate, and colorectal cancer.

### Embodiment 38

The method of any one of the preceding embodiments, wherein the method comprises determining the affinity of the TCR for the epitope peptide-HLA complex.

### Embodiment 39

The method of any one of the preceding embodiments, wherein the method comprises determining the avidity of the TCR for the epitope peptide-HLA complex.

### Embodiment 40

The method of any one of the preceding embodiments, wherein the method comprises repeating the contacting the Cell Product with the polypeptide until the Cell Product is exhausted.

### BRIEF DESCRIPTION OF THE DRAWINGS

FIGS. 1A-1D show exemplary schematics for assays used to assess Cell Products. FIG. 1A, Schematic of a neoepitope (neoE)-specific peptide-HLA complex (comPACT) polypeptide, which comprise a neoE peptide covalently linked to the β2-microglobulin polypeptide covalently linked to HLA class I heavy chain polypeptide. FIG. 1B, Schematic of a comPACT coated plate assay, including three exemplary functional outputs. FIG. 1C, Schematic of a comPACT coated bead assay, including three exemplary functional outputs. FIG. 1D, Schematic of a comPACT coated T2 cells assay, including three exemplary functional outputs.

FIGS. 2A-C show dose-dependent cytokine release using a comPACT coated plate assay by F5 T cells and PACT032-TCR.075 T cells. The extent of IFNγ (FIG. 2A), IL2 (FIG. 2B), and TNFα (FIG. 2C) secretion are shown.

FIG. 3 shows dose-dependent CD107a expression by CD8+ PACT032-TCR.075 T cells and CD8+ F5 TCR T cells in a comPACT coated plate assay.

FIGS. 4A-C show dose-dependent Ki67 expression by CD8+ PACT032-TCR.075 T cells and CD8+ F5 TCR T cells (FIG. 4A) and by CD4+ PACT032-TCR.075 T cells and CD4+ F5 TCR T cells (FIG. 4B) in a comPACT coated plate assay. FIG. 4C shows dose-dependent cell proliferative activity by PACT032-TCR.075 T cells and F5 TCR T cells in a comPACT coated plate assay.

FIGS. 5A-B show dose-dependent IFNγ secretion by Neo12 TCR T cells and F5 TCR T cells following stimulation with comPACTs bound to either 1 micron beads (FIG. 5A) or 6 micron beads (FIG. 5B) in a comPACT coated bead assay.

FIGS. 6A-C show dose-dependent IFNγ (FIG. 6A), IL2 (FIG. 6B), and TNFα (FIG. 6C) secretion by Neo12 TCR T cells following stimulation with or without comPACTs bound to 25,000 T2 cells or to 50,000 T2 cells in a comPACT coated T2 cell assay. The bars in each of FIGS. 6A-6C, from left to right, are 25,000 T2 cells+comPACT, 50,000 T2 cells+comPACT, 25,000 T2 cells with no comPACT, 50,000 T2 cells with no comPACT.

FIGS. 7A-B show dose-dependent IFNγ (FIG. 7A) and TNFα (FIG. 7B) secretion by Neo12 TCR T cells in a comPACT coated plate assay with Tween20 (“PBST”) or without Tween (“PBS”) in the wash buffer.

FIGS. 8A-B show dose-dependent IFNγ secretion by Neo12 TCR T cells following stimulation with K562 cells expressing HLA-A02 or with negative control cells described in Example 12 in a cell-based assay (FIG. 8A) and following stimulation with comPACTs in a comPACT coated plate assay (FIG. 8B).

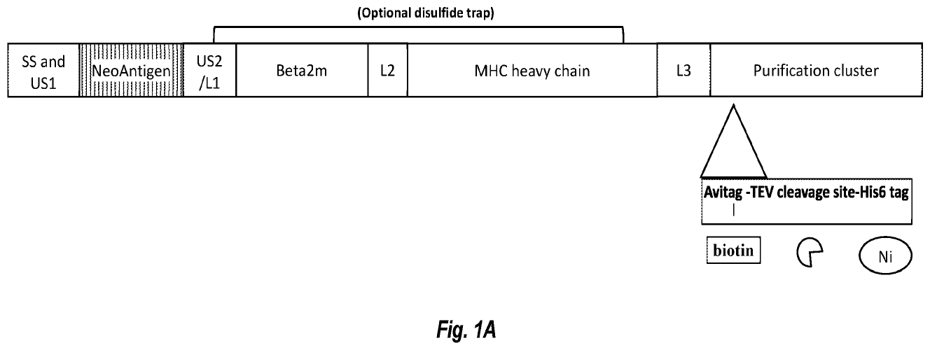

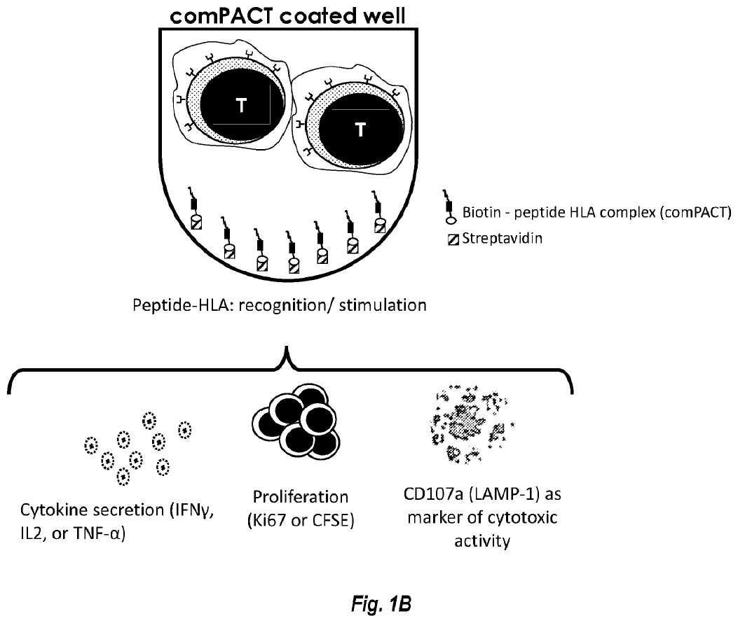

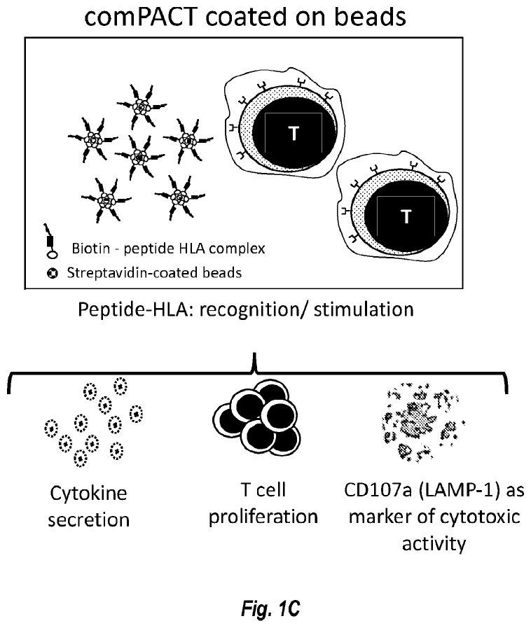

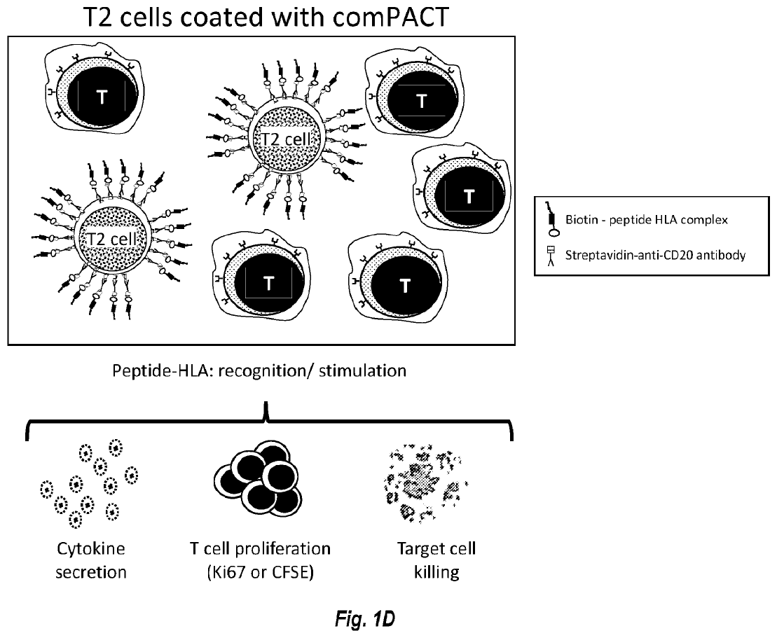

### DETAILED DESCRIPTION

The present disclosure is related to the use of comPACT polypeptides to quantitatively evaluate Cell Products. The present methods may be used, for example, to assay a Cell Product for efficacy, potency, and/or specificity. In some embodiments, the assay is quantitative.

### Definitions

Unless defined otherwise, all technical and scientific terms used herein have the meaning commonly understood by a person skilled in the art. The following references provide one of skill with a general definition of many of the terms used in the presently disclosed subject matter: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

It is understood that aspects and embodiments of the invention described herein include “comprising,” “consisting,” and “consisting essentially of” aspects and embodiments. The terms “comprises” and “comprising” are intended to have the broad meaning ascribed to them in U.S. Patent Law and can mean “includes,” “including” and the like.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold or within 2-fold, of a value.

“Checkpoint Inhibitor” as used herein means a type of drug that blocks certain proteins made by certain types of immune system cells (e.g., T cells) and a subset of cancer cells. Such proteins that are made by certain immune and cancer cells help keep immune responses in check and can keep T cells from killing cancer cells. Accordingly, when these proteins are blocked by a checkpoint inhibitor, T cells are able to kill certain cancer cells. A checkpoint inhibitor is an immunotherapy and the terms are not mutually exclusive as used herein.

The terms “Cancer” and “Tumor” are used interchangeably herein. As used herein, the terms “Cancer” or “Tumor” refer to all neoplastic cell growth and proliferation, whether malignant or benign, and all pre-cancerous and cancerous cells and tissues. The terms are further used to refer to or describe the physiological condition in mammals that is typically characterized by unregulated cell growth/proliferation. Cancer can affect a variety of cell types, tissues, or organs, including but not limited to an organ selected from bladder, bone, brain, breast, cartilage, glia, esophagus, fallopian tube, gallbladder, heart, intestines, kidney, liver, lung, lymph node, nervous tissue, ovaries, pancreas, prostate, skeletal muscle, skin, spinal cord, spleen, stomach, testes, thymus, thyroid, trachea, urogenital tract, ureter, urethra, uterus, and vagina, or a tissue or cell type thereof. Cancer includes cancers, such as sarcomas, carcinomas, or plasmacytomas (malignant tumor of the plasma cells). Examples of cancer include, but are not limited to, those described herein. The terms “Cancer” or “Tumor” and “Proliferative Disorder” are not mutually exclusive as used herein.

“comPACT” and “comPACT polypeptide” are used interchangeably herein and mean a peptide comprising an antigen or epitope amino acid sequence (“epitope peptide”), a J32-microglobulin protein sequence, and an extracellular domain sequence of an MHC heavy chain. The β2-microglobulin protein sequence and extracellular domain sequence of the MHC heavy chain are together herein referred to as an “HLA complex” when they are correctly folded, including, e.g., proper formation of intramolecular disulfide bonds. In some embodiments, the antigen or epitope is a neoantigen or neoepitope. In some embodiments, the β2-microglobulin protein sequence is a full-length β2-microglobulin protein sequence. In some embodiments, the extracellular domain of an MHC heavy chain is the extracellular domain of a class I MHC heavy chain comprising α1, α2, and α3 domains. In some embodiments, the comPACT polypeptide comprises a binding moiety conjugation site and/or a binding moiety conjugated to the site. In some such embodiments, the comPACT polypeptide comprises a biotin conjugation site, e.g., an AviTag and/or a biotin conjugated to the site. In some embodiments, the comPACT polypeptide comprises a cleavable tag and a purification tag, such as a TEV tag and histidine tag, respectively. In some embodiments, the comPACT polypeptide comprises a signal sequence, such as an N-terminal signal sequence. In some embodiments, the comPACT polypeptide comprises one or more linkers between the antigen or epitope sequence, the β2-microglobulin protein sequence, the extracellular domain sequence of the MHC heavy chain, and/or a biotin conjugation site sequence.

“Dextramer” as used herein means a multimerized neoepitope-HLA complex that specifically binds to its cognate NeoTCR.

“Endogenous” as used herein refers to a nucleic acid molecule or polypeptide that is normally expressed in a cell or tissue.

“Exogenous” as used herein refers to a nucleic acid molecule or polypeptide that is not endogenously present in a cell. The term “exogenous” would therefore encompass any recombinant nucleic acid molecule or polypeptide expressed in a cell, such as foreign, heterologous, and over-expressed nucleic acid molecules and polypeptides. By “exogenous” nucleic acid is meant a nucleic acid not present in a native wild-type cell; for example, an exogenous nucleic acid may vary from an endogenous counterpart by sequence, by position/location, or both. For clarity, an exogenous nucleic acid may have the same or different sequence relative to its native endogenous counterpart; it may be introduced by genetic engineering into the cell itself or a progenitor thereof, and may optionally be linked to alternative control sequences, such as a non-native promoter or secretory sequence.

“Immunotherapy” or “Cancer Immunotherapy” as used herein means a therapy designed to treat a disease such as cancer by activating or suppressing the immune system. Immunotherapies can be designed to elicit or amplify an immune response (i.e., activation immunotherapies) or to reduce or suppress an immune response (i.e., suppression immunotherapies). A checkpoint inhibitor is an immunotherapy and the terms are not mutually exclusive as used herein.

“Neoantigen”, “neoepitope” or “neoE” refer to a newly formed antigenic determinant that arises, e.g., from a somatic mutation(s) and is recognized as “non-self.” A mutation giving rise to a “neoantigen”, “neoepitope” or “neoE” can include a frameshift or non-frameshift indel, missense or nonsense substitution, splice site alteration (e.g., alternatively spliced transcripts), genomic rearrangement or gene fusion, any genomic or expression alterations, or any post-translational modifications.

“NeoTCR” as used herein mean a neoepitope-specific T cell receptor that is introduced into a T cell, e.g., by gene editing methods. As used herein, the term “TCR gene sequence” refers to a NeoTCR gene sequence.

“NeoTCR cells” as used herein means a plurality of cells precision engineered to express one or more NeoTCRs. In certain embodiments, the cells are T cells. In certain embodiments, the T cells are CD8+ and/or CD4+ T cells. In certain embodiments, the CD8+ and/or CD4+ T cells are autologous cells from the patient for whom a NeoTCR Product will be administered. The terms “NeoTCR cells” and “NeoTCR-P1 T cells” and “NeoTCR-P1 cells” are used interchangeably herein. In some embodiments, the NeoTCR cells do not comprise any exogenous DNA sequences, e.g., in the genome of the T cells.

“NeoTCR Product” as used herein means a pharmaceutical formulation comprising a plurality of NeoTCR cells. A NeoTCR Product consists of autologous precision genome-engineered CD8+ and CD4+ T cells. Using a targeted DNA-mediated non-viral precision genome engineering approach, expression of the endogenous TCR is eliminated and replaced by a patient-specific NeoTCR isolated from peripheral CD8+ T cells targeting the tumor-exclusive neoepitope. In certain embodiments, the resulting engineered CD8+ and/or CD4+ T cells express NeoTCRs on their surface of native sequence, native expression levels, and native TCR function. The sequences of the NeoTCR external binding domain and cytoplasmic signaling domains are unmodified from the TCR isolated from native CD8+ T cells. Regulation of the NeoTCR gene expression is driven by the native endogenous TCR promoter positioned upstream of where the NeoTCR gene cassette is integrated into the genome. Through this approach, native levels of NeoTCR expression are observed in unstimulated and antigen-activated T cell states. In some embodiments, the NeoTCR Product does not comprise any exogenous DNA sequences, e.g., in the genome of the T cells.

The NeoTCR Product manufactured for each patient represents a defined dose of autologous CD8+ and/or CD4+ T cells that are precision genome engineered to express a single neoepitope (neoE)-specific TCR cloned from neoE-specific CD8+ T cells individually isolated from the peripheral blood of that same patient.

“NeoTCR Viral product” as used herein has the same definition of NeoTCR Product except that the genome engineering is performed using viral mediated methods.

“Pharmaceutical Formulation” refers to a preparation which is in such form as to permit the biological activity of an active ingredient contained therein to be effective, and which contains no additional components which are unacceptably toxic to a subject to which the formulation would be administered. For clarity, DMSO at quantities used in a NeoTCR Product are not considered unacceptably toxic.

A “subject,” “patient,” or an “individual” for purposes of treatment refers to any animal classified as a mammal, including humans, domestic and farm animals, and zoo, sports, or pet animals, such as dogs, horses, cats, cows, etc. Preferably, the mammal is human.

“Cell Product” as used herein means a pharmaceutical formulation comprising a plurality of cells expressing an engineered TCR or expressing an isolated or selected naturally occurring TCR. In certain embodiments, the Cell Product described herein comprises T cells, NK cells, NKT cells, macrophages, tumor infiltrating lymphocytes (TILs), hematopoietic stem cells (HSCs), cells derived from HSCs, or dendritic/antigen-presenting cells. In certain embodiments, the Cell Product described herein comprises T cells. In certain embodiments, the Cell Product cells are primary cells or are derived from primary cells. In certain embodiments, the Cell Product comprise NeoTCR Cells. In certain embodiments the Cell Product is a NeoTCR Product.

“TCR” as used herein means T cell receptor.

“Treat,” “Treatment,” and “treating” are used interchangeably and as used herein mean obtaining beneficial or desired results including clinical results. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, the NeoTCR Product of the invention are used to delay development of a proliferative disorder (e.g., cancer) or to slow the progression of such disease.

“Tumor antigen” as used herein refers to an antigen (e.g., a polypeptide) that is uniquely or differentially expressed on a tumor cell compared to a normal or non-neoplastic cell. In certain embodiments, a tumor antigen includes any polypeptide expressed by a tumor that is capable of activating or inducing an immune response via an antigen-recognizing receptor or capable of suppressing an immune response via receptor-ligand binding.

### Cell Products

In some embodiments, methods herein are useful in evaluating the efficacy of a previously developed Cell Product. In some embodiments, methods herein are useful for screening a Cell Product, thereby aiding in the development of the Cell Product. In some embodiments, methods herein are useful both in the development of a Cell Product and the evaluation of a developed Cell Product. The Cell Product may be any Cell Product comprising T cells expressing a TCR. Nonlimiting exemplary Cell Products are described herein, and include NeoTCRs.

In certain embodiments, the Cell Products used in the methods herein are developed by engineering human T cells. In certain embodiments, such engineering involves genome editing. For example, but not by way of limitation, such genome editing can be accomplished with nucleases targeting one or more endogenous loci, e.g., TCR alpha (TCRα) locus and TCR beta (TCRβ) locus. In certain embodiments, the nucleases can generate single-stranded DNA nicks or double-stranded DNA breaks in an endogenous target sequence. In certain embodiments, the nuclease can target coding or non-coding portions of the genome, e.g., exons, introns. In certain embodiments, the nucleases contemplated herein comprise homing endonuclease, meganuclease, megaTAL nuclease, transcription activator-like effector nuclease (TALEN), zinc-finger nuclease (ZFN), and clustered regularly interspaced short palindromic repeats (CRISPR)/Cas nuclease. In certain embodiments, the nucleases can themselves be engineered, e.g., via the introduction of amino acid substitutions and/or deletions, to increase the efficiency of the cutting activity.

In certain embodiments, a CRISPR/Cas nuclease system is used to engineer human cells to make a cell therapy, such as a Cell Product. In certain embodiments, the CRISPR/Cas nuclease system comprises a Cas nuclease and one or more RNAs that recruit the Cas nuclease to the endogenous target sequence, e.g., single guide RNA. In certain embodiments, the Cas nuclease and the RNA are introduced in the cell separately, e.g. using different vectors or compositions, or together, e.g., in a polycistronic construct or a single protein-RNA complex. In certain embodiments, the Cas nuclease is Cas9 or Cas12a. In certain embodiments, the Cas9 polypeptide is obtained from a bacterial species including, without limitation, Streptococcus pyogenes or Neisseria menengitidis. Additional examples of CRISPR/Cas systems are known in the art. See Adli, Mazhar. “The CRISPR tool kit for genome editing and beyond.” Nature communications vol. 9, 1 1911 (2018), herein incorporated by reference for all that it teaches.

In certain embodiments, genome editing occurs at one or more genome loci that regulate immunological responses. In certain embodiments, the loci include, without limitation, TCR alpha (TCRα) locus, TCR beta (TCRβ) locus, TCR gamma (TCRγ), and TCR delta (TCRδ).

In certain embodiments, genome editing is performed by using non-viral delivery systems. For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically.

In certain embodiments, genome editing is performed by using viral delivery systems. In certain embodiments, the viral methods include targeted integration (including but not limited to AAV) and random integration (including but not limited to lentiviral approaches). In certain embodiments, the viral delivery would be accomplished without integration of the nuclease. In such embodiments, the viral delivery system can be Lentiflash or another similar delivery system.

### NeoTCR Products.

In some embodiments, the Cell Product is a NeoTCR Product. In some embodiments, using the gene editing technology and NeoTCR isolation technology described in PCT/US2020/17887 and PCT/US2019/025415, which are incorporated herein in their entireties, NeoTCRs are cloned in autologous CD8+ and CD4+ T cells from the same patient with cancer by precision genome engineering to express the NeoTCR. In such embodiments, the NeoTCRs that are tumor specific are identified in cancer patients, such NeoTCRs are then cloned, and then the cloned NeoTCRs are inserted into the cancer patient's T cells. NeoTCR expressing T cells are then expanded in a manner that preserves “young” T cell phenotypes, resulting in a NeoTCR-P1 product (i.e., a NeoTCR Product) in which the majority of the T cells exhibit T memory stem cell (T_{MSC}) and T central memory (T_{CM}) phenotypes. In some embodiments, for example, as a result of the precision genome engineering provided herein, the NeoTCR Product does not comprise any exogenous DNA sequences, e.g., in the genome of the T cells.

These ‘young’ or ‘younger’ or less-differentiated T cell phenotypes confer improved engraftment potential and prolonged persistence post-infusion, for example, in mouse models and in clinical trials of engineered CAR-T cells in patients with hematologic malignancies. Thus, the administration of a NeoTCR Product, consisting significantly of ‘young’ or ‘younger’ T cell phenotypes, has the potential to benefit patients with cancer, through improved engraftment potential, prolonged persistence post-infusion, and rapid differentiation into effector T cells to eradicate tumor cells throughout the body.

Ex vivo mechanism-of-action studies performed with a NeoTCR Product manufactured with T cells from patients with cancer showed comparable gene editing efficiencies and functional activities, as measured by antigen-specificity of T cell killing activity, proliferation, and cytokine production, were observed, demonstrating that the manufacturing process was successful in generating products with T cells from patients with cancer as starting material.

In certain embodiments, the NeoTCR Product manufacturing process involves electroporation of dual ribonucleoprotein species of CRISPR-Cas9 nucleases bound to guide RNA sequences, with each species targeting the genomic TCRa and the genomic TCRP loci. The specificity of targeting Cas9 nucleases to each genomic locus has been previously described in the literature as being highly specific. Comprehensive testing of the NeoTCR Product was performed in vitro and in silico analyses to survey possible off-target genomic cleavage sites, using COSMID and GUIDE-seq, respectively. Multiple NeoTCR Product or comparable cell products from healthy donors were assessed for cleavage of the candidate off-target sites by deep sequencing, supporting the published evidence that the selected nucleases are highly specific.

Further aspects of the precision genome engineering process have been assessed for safety. No evidence of genomic instability following precision genome engineering was found in assessing multiple NeoTCR Products by targeted locus amplification (TLA) or standard FISH cytogenetics. No off-target integration anywhere into the genome of the NeoTCR sequence was detected. No evidence of residual Cas9 was found in the cell product.

The comprehensive assessment of the NeoTCR Product and precision genome engineering process indicates that the NeoTCR Product will be well tolerated following infusion back to the patient.

The genome engineering approach described herein enables highly efficient generation of bespoke NeoTCR T cells (i.e., NeoTCR Products) for personalized adoptive cell therapy for patients with solid and liquid tumors. Furthermore, the engineering method is not restricted to the use in T cells and has also been applied successfully to other primary cell types, including natural killer and hematopoietic stem cells.

### Tumor-Infiltrating Lymphocytes and CAR-T Cells

In some embodiments, the Cell Product comprises an engineered TCR. In some embodiments, the Cell Product comprises a naturally occurring TCR, such as a TCR produced by a subject or patient. In some embodiments, the Cell Product is a tumor-infiltrating lymphocyte (TIL) product. In some such embodiments, the Cell Product is an enriched and expanded population of tumor-infiltrating lymphocytes. In some embodiments, the Cell Product is a chimeric antigen receptor (CAR)-Cell Product. In some embodiments, the epitope peptide sequence and/or MHC class or HLA type of the polypeptide, such as the comPACT polypeptide, is determined by a method comprising sequencing the TCR of the Cell Product. In some embodiments, the polypeptide, such as a comPACT polypeptide, is selected or designed based on a polypeptide comprising an epitope peptide sequence used in the development or selection of the Cell Product.

Epitope-HLA Complex Polypeptides, Such as comPACT Polypeptides

Methods herein comprise contacting cells of a Cell Product with a polypeptide that comprises an epitope peptide sequence, an HLA sequence, and a β2-microglobulin sequence. In some embodiments, the polypeptide is a comPACT polypeptide. In some embodiments, the HLA sequence is an HLA class I sequence. In some embodiments, the epitope peptide sequence is a neoantigen or neoepitope peptide sequence. In some embodiments, the polypeptide comprises a signal sequence, such as an N-terminal signal sequence, to facilitate secretion of the polypeptide. In some embodiments, the polypeptide comprises a purification tag, such as a histidine tag, which is used to purify the polypeptide following recombinant production of the polypeptide. In some embodiments, the polypeptide comprises a cleavage tag, such as a TEV tag, between the purification tag and the remainder of the polypeptide to facilitate removal of the purification tag prior to contacting a Cell Product with the polypeptide. In some embodiments, the polypeptide comprises a binding moiety conjugation site and/or a binding moiety conjugated to the conjugation site. In some such embodiments, the polypeptide comprises a biotin conjugation site, e.g., an AviTag and/or a biotin conjugated to the site. In some embodiments, the polypeptide comprises one or more linkers between the epitope sequence, the β2-microglobulin sequence, the HLA sequence, and/or a biotin conjugation site sequence. In some embodiments, the Cell Product is a NeoTCR Product. In some embodiments, the cells of a Cell Product are NeoTCR Cells.

In some embodiments, the polypeptide is bound to a solid support. In some such embodiments, the polypeptide comprises or is conjugated to a binding moiety that binds to a binding partner conjugated to the solid support. In some such embodiments, the binding partners on the polypeptide and solid support are a binding pair, such as biotin and streptavidin. In some embodiments, the solid support is a plate, such as a multiwell plate coated with the binding partner of the binding moiety that is conjugated to the polypeptide. In some embodiments, the solid support is a bead, such as a magnetic bead, a streptavidin coated bead, or any type of bead used in a cytometric bead array. In some embodiments, the beads have an average diameter of 1-10 μm. In some embodiments, the solid support is a cell. In some embodiments, the solid support is a T2 cell. In some embodiments, the cell expresses or is coated with a binding partner of the binding moiety conjugated to the polypeptide.

In some embodiments, methods herein comprising contacting a Cell Product with a polypeptide comprising an epitope peptide sequence facilitate improved assay sensitivity, specificity, quantification, reproducibility, and/or convenience and lower cost compared to methods comprising contacting a Cell Product with a cell line expressing an epitope peptide sequence. For example, such embodiments eliminate the need to generate new cell lines to evaluate Cell Products. In some embodiments, the polypeptides used in the methods herein are the same polypeptides used in the production of the Cell Products, thereby further increasing the ease of development of the methods herein relative to alternative methods to evaluate Cell Products. In some embodiments, the Cell Product is a NeoTCR Product. In some embodiments, the cells of a Cell Product are NeoTCR Cells.

### Methods for Evaluating Cell Products

Methods described herein comprise contacting a Cell Product with a polypeptide, such as a comPACT polypeptide, and detecting at least one marker of T cell function. In some embodiments, the methods facilitate high-throughput, quantitative, reproducible, scalable, and/or automatable analysis of the cells of a Cell Product, for example, prior to administration of the Cell Product to a subject or patient. In some embodiments, the Cell Product is a NeoTCR Product. In some embodiments, the cells of a Cell Product are NeoTCR Cells. In some embodiments, the Cell Product is a NeoTCR Product, wherein the same comPACT polypeptide used in the assay methods herein is also used to generate the NeoTCR Product, eliminating the need to generate a target cell line or a new antigen peptide to evaluate the NeoTCR Product. In some embodiments, the comPACT polypeptide is bound to a solid support, such as a plate, beads, or cells. In some embodiments, the methods are easily applied to TCRs restricted on any MHC. In some embodiments, the methods eliminate the need to generate a new cell line to test each Cell Product. In some embodiments, the sensitivity and specificity of the methods are comparable to or improved relative to methods comprising target cells.

### Polypeptide Coated Plate Assay

In some embodiments, a polypeptide, such as a comPACT polypeptide is bound to a plate, such as a multiwell plate. In some such embodiments, the comPACT polypeptide comprises a binding moiety and the plate is coated with a binding partner of the binding moiety. For example, in some embodiments, the comPACT polypeptide comprises a biotin and the plate is coated with streptavidin. Methods comprising such coated plate assays allow control of the concentration of comPACT used in each well and therefore allow quantitative, dose-dependent analysis of the function of the cells of a Cell Product. In some embodiments, streptavidin coated plates are washed with a wash buffer comprising tween, such as tween20, before coating the plate with comPACT polypeptides. In some embodiments, the plate is washed with wash buffer comprising tween, such as tween20, after incubation with the comPACT polypeptides. In some embodiments, the plate is then washed with a buffer that removes the tween. Subsequent steps in methods comprising a comPACT coated plate include adding a Cell Product to the plate and detecting a marker for T cell function. In some embodiments, the Cell Product is a NeoTCR Product. In some embodiments, the cells of a Cell Product are NeoTCR Cells.

### Polypeptide Coated Bead Assay

In some embodiments, a polypeptide, such as a comPACT polypeptide, is bound to a bead, such as a bead with a diameter of 1-10 microns or 1-6 microns. In some such embodiments, the comPACT polypeptide comprises a binding moiety and the bead is coated with a binding partner of the binding moiety. For example, in some embodiments, the comPACT polypeptide comprises a biotin and the bead is coated with streptavidin. In some embodiments, methods comprising such coated bead assays allow for increased contact surface area of the comPACT polypeptides and the Cell Products over alternative methods, resulting in improved sensitivity over alternative methods, such as co-culture methods. In some embodiments, the coated bead assay methods also allow control of the concentration of comPACT used and therefore allow quantitative, dose-dependent analysis of Cell Product function. In some embodiments, the Cell Product is a NeoTCR Product. In some embodiments, the cells of a Cell Product are NeoTCR Cells.

### Polypeptide Coated Cells Assay

In some embodiments, a polypeptide, such as a comPACT polypeptide, is bound to a cell, such as a T2 cell, that is coated with a binding partner of a binding moiety conjugated to the polypeptide. In some such embodiments, the binding partner of the binding moiety conjugated to the polypeptide is conjugated to an antibody that specifically binds to an antigen expressed on the cell surface. Contacting the cell with 1) the binding partner conjugated to antibody that specifically binds to the antigen expressed by the cell and 2) the polypeptide conjugated to the binding moiety that binds the binding partner on the surface of the cell produces polypeptide coated cells. In some embodiments, methods comprising such coated cells allow for increased contact surface area of the polypeptides and the cells of a Cell Product than over alternative methods, resulting in improved sensitivity. In some embodiments, the Cell Product is a NeoTCR Product. In some embodiments, the cells of a Cell Product are NeoTCR Cells.

### Functional Marker Detection

Methods herein comprise detecting at least one marker of T cell function in or expressed by the cells of a Cell Product. In some embodiments, the marker of T cell function is a proliferation marker. In some such embodiments, the proliferation marker is Ki67. In some embodiments, proliferation is measured using carboxyfluorescein succinimidyl ester (CFSE). In some embodiments, the marker of T cell function is a cytotoxicity marker. In some such embodiments, the cytotoxicity marker is CD107a. In some embodiments, the marker of T cell function is an activation marker. In some such embodiments, the activation marker is IFN-γ, IL2, TNFα, CD69, CD44, or CD25. In some embodiments, the Cell Product is a NeoTCR Product. In some embodiments, the cells of a Cell Product are NeoTCR Cells.

In some embodiments, the detecting of at least one marker of T cell function comprises contacting the Cell Product with a binding molecule, such as an antibody, that specifically binds at least one marker of T cell function. In some embodiments, the T cell function marker antibody is conjugated to a label, such as a fluorophore, or a binding moiety. In some embodiments, the detecting comprises performing flow cytometry or a cytometric bead array. In some embodiments, the detecting of at least one marker of T cell function comprises any assay for T cell function marker detection known by one of ordinary skill in the art. In some embodiments, the Cell Product is a NeoTCR Product.

### Assay Specificity and Quantifiability

Methods used to evaluate Cell Products, such as the methods herein, must be specific to ensure that the T cell function detected is due to the interaction between the epitope peptide sequence and the Cell Product. In some embodiments, the methods herein provide specific assessment of Cell Product function. In some embodiments, the Cell Product is a NeoTCR Product. For example, in Examples 5, 7, 8, and 12 herein, comPACT peptides comprising an epitope peptide sequence that is not recognized by the TCR of the NeoTCR T cells (mismatched peptide) showed no observable or significant activation in the presence of the mismatched peptide.

Optimal methods used to evaluate the efficacy of Cell Products are, in some embodiments, quantifiable and reproducible. The concentration of polypeptides with which a cell of a Cell Product is contacted in cell-based methods may be impossible to determine. In contrast, the methods herein are quantifiable and reproducible. Concentration of the polypeptides with which the Cell Products are contacted are easily controlled, and the T cell function signals are dose-dependent, specific, reproducible, and correlate closely with gene editing efficiency. In some embodiments, the methods herein facilitate improved Cell Product evaluation, resulting in improved therapeutic efficacy of Cell Products. In some embodiments, the Cell Product is a NeoTCR Product. In some embodiments, the cells of a Cell Product are NeoTCR Cells.

### Methods of Treatment

The Cell Products and cells thereof evaluated in the assay methods disclosed herein can be used to treat subjects. In some embodiments, a Cell Product that produces an effective signal in a method herein is used to treat a subject. In some embodiments, a Cell Product that does not produce an effective signal in a method herein is not used to treat a subject. In some embodiments, a method provided herein is used to determine the appropriate dose of a Cell Product. In some embodiments, the Cell Product is a NeoTCR Product. In some embodiments, the cells of a Cell Product are NeoTCR Cells.

In certain embodiments, the Cell Products can be administered with a checkpoint inhibitor. In certain embodiments, the Cell Products can be administered with an immunotherapy. In certain embodiments, the immunotherapy is a cancer vaccine. In certain embodiments, the immunotherapy is a cytokine therapy (i.e., cytokines and modifications, derivatives, and fusion proteins thereof that are formulated into a pharmaceutical formulation). In certain embodiments, the immunotherapy is a T cell therapy. In certain embodiments, the immunotherapy is a CAR-T cell therapy. In certain embodiments, the immunotherapy is an interferon. In certain embodiments, the immunotherapy is an interleukin. In certain embodiments, the immunotherapy is an oncolytic virus therapy. In certain embodiments, the immunotherapy is an NK cell therapy. In certain embodiments, the Cell Products can be administered to a subject in combination with any other chemotherapeutic agent that is standard of care or otherwise an acceptable agent for treating such a patient based on factors such as disease, age, medical history, and other factors that a medical physician would consider. In some embodiments, the Cell Product is a NeoTCR Product.

In certain embodiments, an effective amount of the Cell Product is delivered through IV administration. In certain embodiments, the Cell Products are delivered through IV administration in a single administration. In certain embodiments, the Cell Products are delivered through IV administration in multiple administrations. In certain embodiments, the Cell Products are delivered through IV administration in two or more administrations. In certain embodiments, the Cell Products are delivered through IV administration in two administrations. In certain embodiments, the Cell Products are delivered through IV administration in three administrations. In some embodiments, the Cell Product is a NeoTCR Product.

Non-limiting examples of cancer include blood cancers (e.g. leukemias, lymphomas, and myelomas), ovarian cancer, breast cancer, bladder cancer, brain cancer, colon cancer, intestinal cancer, liver cancer, lung cancer, pancreatic cancer, prostate cancer, skin cancer, stomach cancer, glioblastoma, throat cancer, melanoma, neuroblastoma, adenocarcinoma, glioma, soft tissue sarcoma, and various carcinomas (including prostate and small cell lung cancer). Suitable carcinomas further include any known in the field of oncology, including, but not limited to, astrocytoma, fibrosarcoma, myxosarcoma, liposarcoma, oligodendroglioma, ependymoma, medulloblastoma, primitive neural ectodermal tumor (PNET), chondrosarcoma, osteogenic sarcoma, pancreatic ductal adenocarcinoma, small and large cell lung adenocarcinomas, chordoma, angiosarcoma, endotheliosarcoma, squamous cell carcinoma, bronchoalveolarcarcinoma, epithelial adenocarcinoma, and liver metastases thereof, lymphangiosarcoma, lymphangioendotheliosarcoma, hepatoma, cholangiocarcinoma, synovioma, mesothelioma, Ewing's tumor, rhabdomyosarcoma, colon carcinoma, basal cell carcinoma, sweat gland carcinoma, papillary carcinoma, sebaceous gland carcinoma, papillary adenocarcinoma, cystadenocarcinoma, medullary carcinoma, bronchogenic carcinoma, renal cell carcinoma, bile duct carcinoma, choriocarcinoma, seminoma, embryonal carcinoma, Wilms' tumor, testicular tumor, medulloblastoma, craniopharyngioma, ependymoma, pinealoma, hemangioblastoma, acoustic neuroma, oligodendroglioma, meningioma, neuroblastoma, retinoblastoma, leukemia, multiple myeloma, Waldenstrom's macroglobulinemia, and heavy chain disease, breast tumors such as ductal and lobular adenocarcinoma, squamous and adenocarcinomas of the uterine cervix, uterine and ovarian epithelial carcinomas, prostatic adenocarcinomas, transitional squamous cell carcinoma of the bladder, B and T cell lymphomas (nodular and diffuse) plasmacytoma, acute and chronic leukemias, malignant melanoma, soft tissue sarcomas and leiomyosarcomas. In certain embodiments, the neoplasia is selected from blood cancers (e.g. leukemias, lymphomas, and myelomas), ovarian cancer, prostate cancer, breast cancer, bladder cancer, brain cancer, colon cancer, intestinal cancer, liver cancer, lung cancer, pancreatic cancer, prostate cancer, skin cancer, stomach cancer, glioblastoma, and throat cancer. In certain embodiments, the presently disclosed young T cells and compositions comprising thereof can be used for treating and/or preventing blood cancers (e.g., leukemias, lymphomas, and myelomas) or ovarian cancer, which are not amenable to conventional therapeutic interventions.

In certain embodiments, the neoplasia is a solid cancer or a solid tumor. In certain embodiments, the solid tumor or solid cancer is selected from glioblastoma, prostate adenocarcinoma, kidney papillary cell carcinoma, sarcoma, ovarian cancer, pancreatic adenocarcinoma, rectum adenocarcinoma, colon adenocarcinoma, esophageal carcinoma, uterine corpus endometrioid carcinoma, breast cancer, skin cutaneous melanoma, lung adenocarcinoma, stomach adenocarcinoma, cervical and endocervical cancer, kidney clear cell carcinoma, testicular germ cell tumors, and aggressive B-cell lymphomas.

The subjects can have an advanced form of disease, in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence.

Suitable human subjects for therapy typically comprise two treatment groups that can be distinguished by clinical criteria. Subjects with “advanced disease” or “high tumor burden” are those who bear a clinically measurable tumor. A clinically measurable tumor is one that can be detected on the basis of tumor mass (e.g., by palpation, CAT scan, sonogram, mammogram or X-ray; positive biochemical or histopathologic markers on their own are insufficient to identify this population). A pharmaceutical composition is administered to these subjects to elicit an anti-tumor response, with the objective of palliating their condition. Ideally, reduction in tumor mass occurs as a result, but any clinical improvement constitutes a benefit. Clinical improvement includes decreased risk or rate of progression or reduction in pathological consequences of the tumor.

### Compositions and Articles of Manufacture

The Cell Products described herein can be used in combination with articles of manufacture. In some embodiments, the Cell Product is a NeoTCR Product. Such articles of manufacture can be useful for the prevention or treatment of proliferative disorders (e.g., cancer). Examples of articles of manufacture include but are not limited to containers (e.g., infusion bags, bottles, storage containers, flasks, vials, syringes, tubes, and IV solution bags) and a label or package insert on or associated with the container. The containers may be made of any material that is acceptable for the storage and preservation of the NeoTCR Cells within the NeoTCR Products. In certain embodiments, the container may be an intravenous solution bag or a vial having a stopper pierceable by a hypodermic injection needle. For example, the container may be a CryoMACS freezing bag. The label or package insert indicates that the NeoTCR Products are used for treating the condition of choice and the patient of origin. The patient is identified on the container of the NeoTCR Product because the NeoTCR Products is made from autologous cells and engineered as a patient-specific and individualized treatment.

The article of manufacture may comprise a container with one NeoTCR Product contained therein. The article of manufacture may comprise a container with two NeoTCR Products contained therein. The article of manufacture may comprise a container with three NeoTCR Products contained therein. The article of manufacture may comprise a container with four NeoTCR Products contained therein. The article of manufacture may comprise a container with five NeoTCR Products contained therein.

Furthermore, any container of NeoTCR Product described herein can be split into two, three, or four separate containers for multiple time points of administration and/or based on the appropriate dose for the patient.

In certain embodiments, the NeoTCR Products are provided in a kit. The kit can, by means of non-limiting examples, contain package insert(s), labels, instructions for using the NeoTCR Product(s), syringes, disposal instructions, administration instructions, tubing, needles, and anything else a clinician would need in order to properly administer the NeoTCR Product(s).

The NeoTCR Products disclosed herein are formulated into pharmaceutical formulation for the use thereof. In certain embodiments, the label of such NeoTCR Products provides instructions for treating a subject in need thereof.

### Therapeutic Compositions and Methods of Manufacturing

The Cell Products described herein can be formulated into a drug product using the clinical manufacturing process. In some embodiments, the Cell Product is a NeoTCR Product. In some embodiments and under this clinical manufacturing process, the NeoTCR Products are cryopreserved in CryoMACS Freezing Bags or another suitable cryobag. In some embodiments, one or more bags may be shipped to the site for each patient depending on patient needs. In some embodiments, the product is composed of apheresis-derived, patient-autologous, CD8 and CD4 T cells that have been precision genome engineered to express one or more autologous neoTCRs targeting a neoepitope complexed to one of the endogenous HLA receptors presented exclusively on the surface of that patient's tumor cells.

### Kits

The presently disclosed subject matter provides kits for evaluating a Cell Product and/or screening a Cell Product, and/or treating and/or preventing a cancer or a pathogen infection in a subject. In certain embodiments, the kit comprises an effective amount of a Cell Product or a pharmaceutical composition comprising thereof. In some embodiments, the kit comprises one or more polypeptides, such as comPACT polypeptides, comprising an epitope peptide sequence. In some embodiments, the kit comprises one or more nucleic acids, such as one or more vectors, encoding for the one or more polypeptides, such as comPACT polypeptides. In some embodiments, the kit comprises reagents used in detection of one or more markers of T cell function. In some embodiments, the kit comprises a molecule that detects a marker of T cell function. In some embodiments, the kit comprises a Cell Product. In certain embodiments, the kit comprises a sterile container; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments. In certain non-limiting embodiments, the kit includes an isolated nucleic acid molecule encoding a presently disclosed HR template. In some embodiments, the Cell Product is a NeoTCR Product.

If desired, the Cell Product and/or polypeptides nucleic acid molecules are provided together with instructions for evaluating and/or administering the Cell Product to a subject having or at risk of developing a cancer or pathogen or immune disorder. The instructions generally include information about the use of the composition for the treatment and/or prevention of a cancer or a pathogen infection. In certain embodiments, the instructions include at least one of the following: description of the therapeutic agent; dosage schedule and administration for treatment or prevention of a neoplasia, pathogen infection, or immune disorder or symptoms thereof; precautions; warnings; indications; counter-indications; over-dosage information; adverse reactions; animal pharmacology; clinical studies; and/or references. The instructions may be printed directly on the container (when present), or as a label applied to the container, or as a separate sheet, pamphlet, card, or folder supplied in or with the container. The resulting cells can be grown under conditions similar to those for unmodified cells, whereby the modified cells can be expanded and used for a variety of purposes. In some embodiments, the Cell Product is a NeoTCR Product.

It will be readily apparent to those skilled in the art that other suitable modifications and adaptations of the methods described herein may be made using suitable equivalents without departing from the scope of the embodiments disclosed herein. Having now described certain embodiments in detail, the same will be more clearly understood by reference to the following examples, which are included for purposes of illustration only and are not intended to be limiting. The scope of the disclosure is indicated by the appended claims rather than by the foregoing description, and all changes that come within the meaning and range of equivalency of the claims are therefore intended to be embraced herein.

### EXAMPLES

The following are examples of methods and compositions of the invention. It is understood that various other embodiments may be practiced, given the general description provided above.

### Example 1: Materials and Methods

Generation of NeoTCR T Cells. Neo12, F5, and PACT032-TCR.075 neoepitope-specific TCR T cells were generated as described in PCT/US18/058230. NeoTCR T cells specific for individual patients can be prepared, e.g., as described in PCT Publication Nos. WO 2020/0167918, WO 2019/089610, and/or WO 2019/195310. The assays described herein are suitable for use with any neoepitope-specific TCR T cells and other types of engineered T cells.

comPACT polypeptides. Biotinylated neoE (neoepitope)-specific peptide-HLA complex (comPACT) polypeptides, which comprise a neoE peptide covalently linked to the J32-microglobulin polypeptide covalently linked to HLA class I heavy chain polypeptide, were prepared as described in PCT Publication Nos. WO 2020/0167918 and WO 2019/195310. Personalized neoE-HLA comPACT polypeptides, as defined by the tumor mutations and the HLA types identified for each respective subject, are developed for each patient. comPACT polypeptides shown in the table below were used in the exemplary validation experiments described herein. Neo12 and MART-1 epitope peptides were purchased from Bio-Synthesis or Genscript and resuspended in DMSO at a concentration of 10 mM.

TABLE 1
HLA and epitope peptide sequences for exemplary
comPACTs

| comPACT name | HLA | Epitope Peptide |
|---|---|---|
| Neo12 comPACT | HLA-A02 | YLYHRVDVI |
| MART1 comPACT | HLA-A02 | ELAGIGILTV |
| CMV comPACT | HLA-A02 | NLVPMVATV |
| HLA-A31 comPACT | HLA-A31 | KLRRNANSR |
| HLA-B50 comPACT | HLA-B50 | SEFLVGSP |

comPACT coated plate assay. Streptavidin was diluted in 0.1M NaHCO₃ and added to 96-well plates at 100 μL/well, followed by overnight incubation at 4° C., or commercially available streptavidin coated 96-well plates were used. Streptavidin coated plates were washed 3 times with wash buffer (PBS with 1% BSA and 0.05% Tween20), then 100 μL of comPACT at a concentration ranging from 10 μg/mL to 0.00005 μg/mL was added to each well. Wells with no comPACT and wells coated with mismatch comPACT were used as negative controls. The plates were incubated for 2 hours at room temperature, then washed 3 times with wash buffer and 3 times with TexMACS supplemented with 3% human AB serum to remove the Tween20. NeoTCR-P1 T cells were counted and washed twice with media, then resuspended at 1 million viable nucleated cells/mL in TexMACS (Miltenyi Biotec) supplemented with 3% human AB serum and a penicillin-streptomycin solution. Protein transport inhibitors containing either Monensin and/or Brefeldin A were also added as appropriate. For CD107a staining, 100 μL/well anti-CD107a antibody solution was added. The T cells were plated onto the comPACT coated plate at 100 μL/well and incubated at 37° C., 5% CO₂ for a set period of time.

comPACT coated bead assay. Streptavidin coated beads with a diameter of 1-6 microns were incubated with 20 μmol comPACT in 2.5 μL 8 M bead stock, resuspended in PBS to reach a final volume of 100 μL, for 30 minutes at room temperature using gentle rotation to prevent bead settling. After the incubation, the solution was bound to a magnet rack for 2 or 3 minutes, washed 3 or 4 times with PBS, and then resuspended in 50 μL PBS. comPACT coated beads were then incubated with NeoTCR-P1 T cells at a ratio of 3:1 (300,000 beads to 100,000 cells) for a set period of time.

comPACT coated T2 cells assay. T2 cells were labelled with streptavidin conjugated anti-CD20 antibody for 30 minutes at 4° C. Excess streptavidin conjugated anti-CD20 antibody was removed by washing two times with Pharmingen stain buffer (neutral pH (pH 7.4)-buffered salt solution (i.e., DPBS) that was supplemented with 0.2% (w/v) bovine serum albumin (BSA). comPACT was added at 10 μg/mL for 2 hours at 4° C. Excess comPACT was removed by washing two times with Pharmingen stain buffer. The NeoTCR-P1 cells were then added for a set period of time.

Cytokine release. NeoTCR-P1 cells were added to plates, as described in the comPACT coated plate, bead, or T2 assay protocols, and incubated at 37° C., 5% CO₂ for 24 hours. Supernatants were collected and stored at −80° C. until ready for analysis. Cytokine secretion, such as IFN-γ, TNFα, IL2 or IL6 secretion, was measured using a cytometric bead array (CBA) (Human Th1/Th2 Cytokine kit, BD Bioscience).

Flow cytometry analysis of T cell activation, cytotoxicity, and proliferation markers. NeoTCR-P1 cells were added to plates, as described in the comPACT coated plate, bead, or T2 assay protocols, and incubated at 37° C., 5% CO₂ for the appropriate time.

For CD107a staining, cells were incubated overnight with allophycocyanin (APC) labeled anti-CD107a antibody. Following incubation with antibody, the cells were gently pipetted up and down several times and transferred to 96 well U bottom plates. The cells were centrifuged and resuspended in 200 μL Pharmingen Stain Buffer (BD Biosciences) per well, then and centrifuged again and resuspended in 50 μL of Live/Dead NIR fluorescent reactive stain (1:250; Invitrogen) per well. The plates were incubated for 20 minutes at 4° C. in the dark. 150 μL/well of Pharmingen Stain Buffer was added, and plates were centrifuged then resuspended in 100 μL/well of antibody mix comprising antibodies specific for markers such as CD4 and CD8a. Compensation beads (e.g., Ultra Comp eBeads, Thermo Fisher Scientific) were used as positive controls. The plates were incubated for 20 minutes at 4° C. in the dark, then 100 μL Pharmingen Stain Buffer was added to each well for a total volume of approximately 200 μL per well. The plates were centrifuged, the cells were resuspended in 200 μL Pharmingen Stain Buffer per well, and the plates were centrifuged again.

For CD107a staining, the cells were resuspended in 100 μL/well of IC fixation buffer and incubated at 4° C. in the dark until ready for analysis on the flow cytometer. Before acquisition on the flow cytometer, 125 μL/well of BD Pharmingen Stain Buffer was added and the cells were thoroughly pipetted.

For Ki67 staining following incubation of the cells on the comPACT coated plate, the cells were gently pipetted up and down several times and transferred to 96 well U bottom plates. The cells were centrifuged and resuspended in 200 μL Pharmingen Stain Buffer (BD Biosciences) per well, then and centrifuged again and resuspended in 50 μL of Live/Dead NIR fluorescent reactive stain (1:250; Invitrogen) per well. The plates were incubated for 20 minutes at 4° C. in the dark. 150 μL/well of Pharmingen Stain Buffer was added, and plates were centrifuged then resuspended in 100 μL/well of antibody mix comprising antibodies specific for markers such as CD4 and CD8a. Compensation beads (e.g., Ultra Comp eBeads, Thermo Fisher Scientific) were used as positive controls. The plates were incubated for 20 minutes at 4° C. in the dark, then 100 μL Pharmingen Stain Buffer was added to each well for a total volume of approximately 200 μL per well. The plates were centrifuged, the cells were resuspended in 200 μL Pharmingen Stain Buffer per well, and the plates were centrifuged again and the supernatant was aspirated carefully. 200 μL of Foxp3 Fixation/Permeabilization solution (ThermoFisher) was added to each well and pipetted up and down several times. Plates were incubated for 30 minutes at 4° C. in the dark, centrifuged, and 200 μL Permeabilization Buffer was added to each well. Plates were centrifuged again, and cells were resuspended in 100 μL/well anti-Ki67 antibody diluted in Permeabilization Buffer at 1:100. The cells were pipetted up and down several times and incubated for 30 minutes at 4° C. in the dark. 100 μL of 1× Permeabilization Buffer was added to each well, plates were centrifuged, and the cells were resuspended in 200 μL BD Pharmingen Stain Buffer and analyzed by a flow cytometer.

Flow cytometry acquisition was performed on an Attune N×T flow cytometer (Invitrogen).

Software. FlowJo software was used to analyse flow cytometry data, Graphpad Prism used for data representation and statistical analysis, RAWgraph was used for circle packing visualizations.

### Example 2. Gene Editing Efficiency of NeoTCR Cells Correlates with the Extent of Cytokine Secretion in comPACT Coated Plate Assay

To assess the IFNγ limit of detection in the comPACT coated plate assay, Neo12 T cells with 64% gene editing were generated at laboratory scale. Two-fold serial dilutions were performed by adding CD4/CD8 T cells from the same donors that were activated, electroporated without DNA/RNP and expanded (mock control T cells) and the diluted T cells were tested in the comPACT coated plate assay, as described in Example 1. IFNγ secretion was measured using the cytometric bead array (CBA) cytokine release assay described in Example 1. As shown in Table 2, IFNγ secretion was detected even when Neo12 T cells with 1% gene editing were tested, and the extent of gene editing of the Neo12 T cells correlated with the extent of IFNγ secretion.

TABLE 2
IFNγ secretion (pg/mL) by Neo12 T cells stimulated by comPACT

| Percent of | | | | | | |
|---|---|---|---|---|---|---|
| Neo12 T | | | | | | |
| cells in T | | 0.01 | | | | 100 |
| cell | No | ng/well | 0.1 ng/well | 1 ng/well | 10 ng/well | ng/well |
| population | comPACT | comPACT | comPACT | comPACT | comPACT | comPACT |
| Mock (0%) | 0 | 0 | 0 | 0 | 0 | 0 |
| 1% | 0 | 0 | 0 | 240.9 | 446.9 | 314.5 |
| 2% | 0 | 0 | 22.8 | 614.0 | 760.3 | 675.2 |
| 4% | 0 | 0 | 183.3 | 1157.4 | 1550.6 | 1640.3 |
| 8% | 0 | 0 | 391.5 | 2021.0 | 2732.3 | 2121.3 |
| 16% | 0 | 2.3 | 729.7 | 3309.6 | 4480.7 | 4235.8 |
| 32% | 0 | 120.5 | 2332.5 | 5217.4 | 6091.6 | 5986.9 |
| 64% | 0 | 623.2 | 4318.7 | 6703.2 | 6896.8 | 6911.0 |

### Example 3. comPACT Coated Plate Assay is Specific

Specificity of the comPACT coated plate assay was confirmed by testing F5 (MART1 TCR) T cells against different concentrations of matched and unmatched comPACTs coated on the plate, as described in Example 1. IFNγ cytokine release was measured as described in Example 1. As shown in Table 3, strong IFNγ secretion was detected when F5 T cells were stimulated with matched comPACTs (MART1 HLA-A02). Importantly, no activity was observed when F5 T cells were tested in presence of comPACTs comprising unmatched peptides or HLAs, even at 1000 ng/well.

TABLE 3
IFNγ secretion (pg/mL) by F5 (MART1
TCR) T cells stimulated by comPACT

| comPACT | Neo12- | MART1- | CMV- | HLA-A31 | HLA-B50 |
|---|---|---|---|---|---|
| (ng) | comPACT | comPACT | comPACT | comPACT | comPACT |
| 1000 | 0 | 5234.5 | 0 | 0 | 0 |
| 100 | 0 | 5592.5 | 0 | 0 | 0 |
| 10 | 0 | 5241.8 | 0 | 0 | 0 |
| 1 | 0 | 1155.2 | 0 | 0 | 0 |
| 0.1 | 0 | 0 | 0 | 0 | 0 |
| 0.01 | 0 | 0 | 0 | 0 | 0 |

### Example 4. The Number of T Cells Per Well Correlates with the Extent of Cytokine Secretion in comPACT Coated Plate Assay

To determine the optimal range of NeoTCR T cell numbers in the comPACT coated plate assay, increasing numbers of Neo12 T cells with 19% gene editing efficiency were added in different comPACT-coated wells, and the assay was performed and IFNγ secretion was measured as described in Example 1. As shown in Table 4, the amount of IFNγ detected in the supernatant increased with increasing numbers of total cells and therefore the number of Neo12 T cells. The higher the number of cells, the higher IFNγ secretion even at low concentrations of comPACT per well. “N.D.” in the table indicates no data.

TABLE 4
IFNγ secretion (pg/mL) by Neo12 T cells stimulated by comPACT

| comPACT | 800,000 | 400,000 | 250,000 | 100,000 | 50,000 | 25,000 | 12,500 |
|---|---|---|---|---|---|---|---|
| (ng/well) | cells/well | cells/well | cells/well | cells/well | cells/well | cells/well | cells/well |
| 100 | 7116.0 | N.D. | 6238.6 | 3691.8 | 2122.3 | 805.8 | 266.5 |
| 10 | 7438.4 | 7494.7 | 5371.0 | 3322.1 | 1888.5 | 594.4 | N.D. |
| 1 | 7333.5 | 6699.7 | 4476.8 | 2336.1 | 900.1 | 363.0 | 132.4 |
| 0.1 | 5861.3 | 2445.0 | 643.4 | 251.2 | 33.2 | 0.0 | 0.0 |
| 0.01 | 0 | 0 | 0 | 0 | 0 | 0 | 0 |

### Example 5. TCR Affinity Correlates with the Extent of Cytokine Secretion in comPACT Coated Plate Assay

T cells expressing TCRs that bind to HLA-A2/MART126-35(A27L) (ELAGIGILTV) MART1 with different affinities, F5 TCR (K_{d}=5.4 μM) and M1W TCR (K_{d}>15 μM), as reported in Bethune et al. Biotechniques, 2017. 62(3): p. 123-130, were tested using the comPACT coated plate assay and IFNγ release assay, as described in Example 1. As shown in Table 5, the extent of IFNγ secretion correlated with the TCR affinity for the comPACT epitope peptide.

TABLE 5
IFNγ secretion (pg/mL) by T cells stimulated by comPACT

| comPACT | F5 T cells | | M1W T cells | |
|---|---|---|---|---|
| Peptide | Replicate | Replicate | Replicate | Replicate |
| (ng/mL) | 1 | 2 | 1 | 2 |
| 2000 | 1492.2 | 1106.3 | 498.1 | 470.7 |
| 200 | 1481.5 | 1004.5 | 501.3 | 439.6 |
| 20 | 1350.2 | 1092.1 | 507.0 | 462.3 |
| 2 | 480.5 | 370.7 | 138.3 | 115.2 |
| 0.2 | 1.7 | 0 | 0 | 0 |
| 0.02 | 0 | 0 | 0 | 0 |
| no comPACT | 0 | 0 | 0 | 0 |
| Mismatched | 0 | 0 | 0 | 0 |
| comPACT | | | | |

### Example 6. Extent of Release of Several Cytokines Correlates with comPACT Concentration in the comPACT Coated Plate Assay

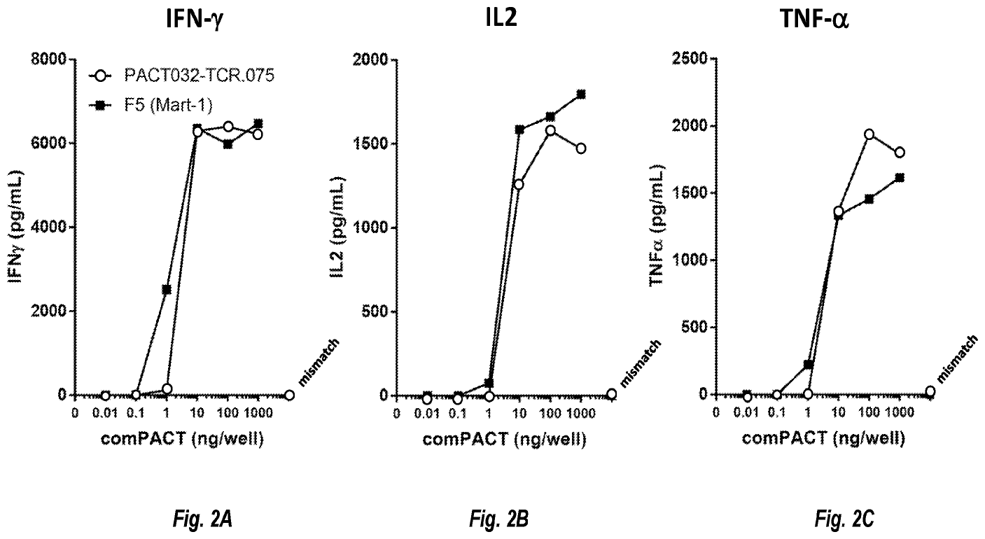

Detection of cytokine release using the comPACT coated plate assay was performed as described in Example 1. F5 T cells and PACT032-TCR.075 T cells were used with their cognate comPACTs, and secretion of several cytokines was measured. As shown in the table below FIGS. 2A-C, the extent of IFNγ (FIG. 2A), IL2 (FIG. 2B), and TNFα (FIG. 2C) secretion correlated with the concentration of cognate comPACT per well in the coated plates.

TABLE 6
Cytokine secretion (pg/mL) by T cells stimulated by comPACT

| | IFNγ (pg/mL) | | IL-2 (pg/mL) | | TNFα (pg/mL) | |
|---|---|---|---|---|---|---|
| comPACT | PACT032- | | PACT032- | | PACT032- | |
| Peptide | TCR.075 T | | TCR.075 T | F5 T | TCR.075 T | F5 T |
| (ng/well) | cells | F5 T cells | cells | cells | cells | cells |
| 1000 | 6215.4 | 6475.1 | 1470.9 | 1795.8 | 1792.1 | 1616.6 |
| 100 | 6404.1 | 5982.3 | 1579.4 | 1662.7 | 1940.3 | 1456.5 |
| 10 | 6297.3 | 6357.7 | 1260.7 | 1585.9 | 1368.6 | 1333.5 |
| 1 | 124.8 | 2517.2 | 0 | 75.3 | 0 | 222.3 |
| 0.1 | 0 | 0 | 0 | 0 | 0 | 0 |
| 0.01 | 0 | 0 | 0 | 0 | 0 | 0 |
| no | 0 | 0 | 0 | 0 | 0 | 0 |
| comPACT | | | | | | |
| Mismatched | 0 | 7 | 0 | 0 | 0 | 0 |
| comPACT | | | | | | |

### Example 7. Cytotoxicity Correlates with comPACT Concentration in the comPACT Coated Plate Assay

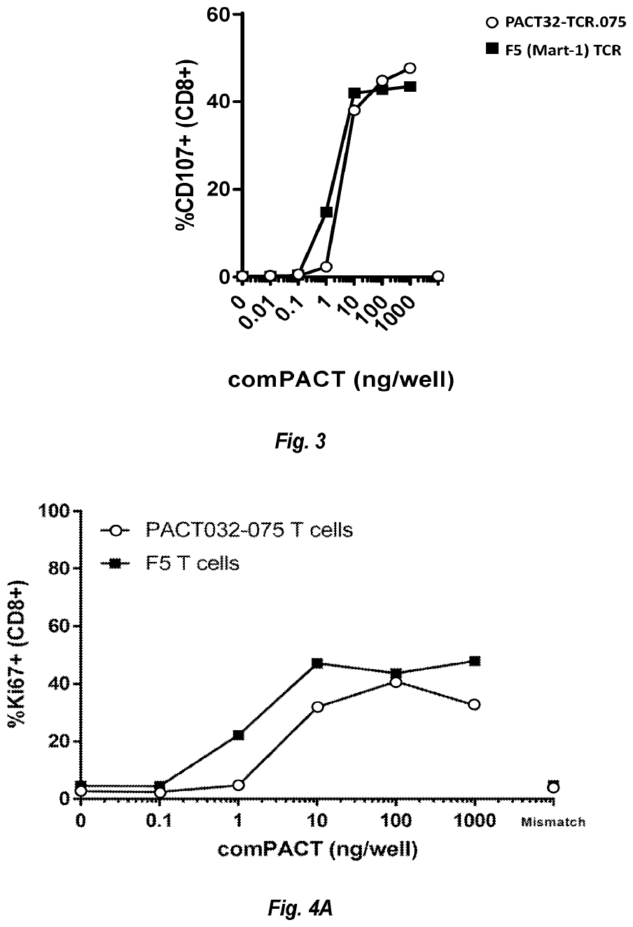

CD107a staining was used to test antigen-specific cytotoxic activity of NeoTCR T cells in the comPACT coated plate assay. Neo12 TCR T cells with 64% gene editing efficiency, PACT032-TCR.075 T cells, and F5 TCR T cells were stimulated with different concentrations of cognate comPACT in the comPACT coated plate assay, followed by CD107a staining and measurement by flow cytometry, as described in Example 1. The results in Table 7 show the percent of CD8+ Neo12 TCR T cells that expressed CD107a. Neo12 TCR T cells showed antigen-specific cytotoxic activity. No activity was observed when mismatched comPACT was used. FIG. 3 shows the percent of CD8+ PACT032-TCR.075 T cells that expressed CD107a and the percent of CD8+ F5 TCR T cells that expressed CD107a. PACT032-TCR.075 T cells and F5 TCR T cells showed antigen-specific cytotoxic activity. No activity was observed when mismatched comPACT was used.

TABLE 7
CD107a expression in Neo12 T cells stimulated by comPACT

| comPACT Peptide (ng/mL) | Percent of cells with CD107a expression |
|---|---|
| 1000 | 76.9 |
| 100 | 75.5 |
| 10 | 74.5 |
| 1 | 33.6 |
| 0.1 | 6.61 |
| 0.01 | 1.22 |
| Mismatch | 1.38 |

### Example 8. Proliferation Correlates with comPACT Concentration in the comPACT Coated Plate Assay

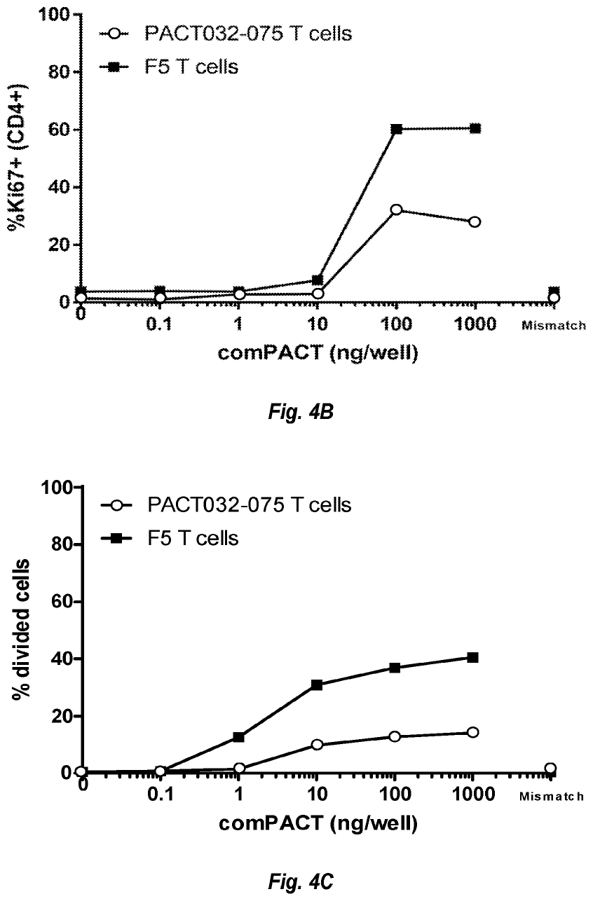

Ki67 staining was used to test antigen-specific proliferative activity of NeoTCR T cells in the comPACT coated plate assay. Expression of Ki67 in F5 TCT T cells and PACT032-TCR.075 TCR T cells was stimulated with different concentrations of cognate comPACT in the comPACT coated plate assay, followed by Ki67 staining and measurement by flow cytometry, as described in Example 1. As shown in FIG. 4, CD4+(FIG. 4B) and CD8+(FIG. 4A) F5 T cells and CD4+(FIG. 4B) and CD8+(FIG. 4A) PACT032-TCR.075 T cells showed antigen-specific proliferative activity. No activity was observed when mismatched comPACT was used.

Staining with e450 proliferation dye was also used to test proliferative activity of F5 TCR T cells and PACT032-TCR.075 TCR T cells in the comPACT coated plate assay. The T cells were stained with e450 proliferation dye and stimulated with different concentrations of cognate comPACT, as described in Example 1, and measurement by flow cytometry. The results, shown in FIG. 4C, show that F5 T cells and PACT032-TCR.075 T cells showed antigen-specific proliferative activity. No activity was observed when mismatched comPACT was used.

### Example 9. Cytokine Release Correlates with comPACT Concentration in the comPACT Coated Bead Assay

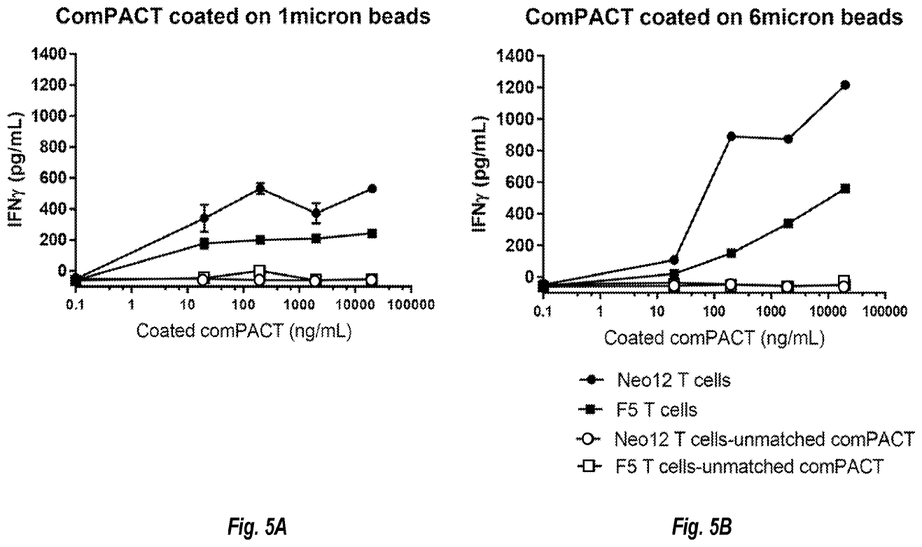

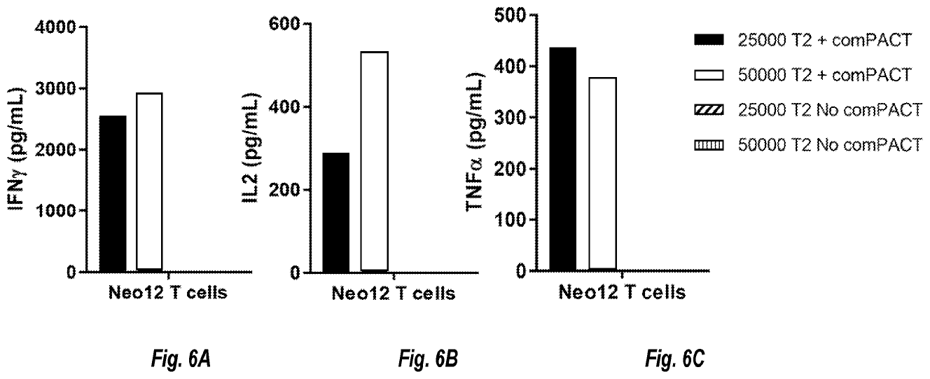

The comPACT coated bead assay was tested by stimulating F5 TCR T cells and Neo12 TCR T cells with different concentrations of matched and unmatched comPACTs coated on beads, as described in Example 1. IFN-γ cytokine release was measured as described in Example 1. As shown in FIG. 5, strong IFN-γ secretion was detected when F5 TCR T cells and Neo12 TCR T cells were stimulated with matched comPACTs bound to either 1 micron beads (FIG. 5A) or 6 micron beads (FIG. 5B). Little to no activity was observed when the cells were tested in presence of comPACTs comprising unmatched peptides, even at over 10,000 ng/well.

### Example 10. Cytokine Release in the comPACT Coated T2 Cells Assay

The comPACT coated T2 cells assay was tested by stimulating Neo12 TCR T cells with different numbers of T2 cells coated with matched comPACTs and T2 cells coated with no comPACT, as described in Example 1. IFN-γ, IL2, and TNFα cytokine release was measured as described in Example 1. As shown in FIG. 6, strong IFN-γ (FIG. 6A), IL2 (FIG. 6B), and TNFα (FIG. 6C) secretion was detected when Neo12 TCR T cells were stimulated with matched comPACTs bound to 25,000 T2 cells or to 50,000 T2 cells. No activity was observed when the cells were tested in presence of T2 cells with no comPACTs bound to them.

### Example 11. Effect of Tween20 in Assay Wash Buffer

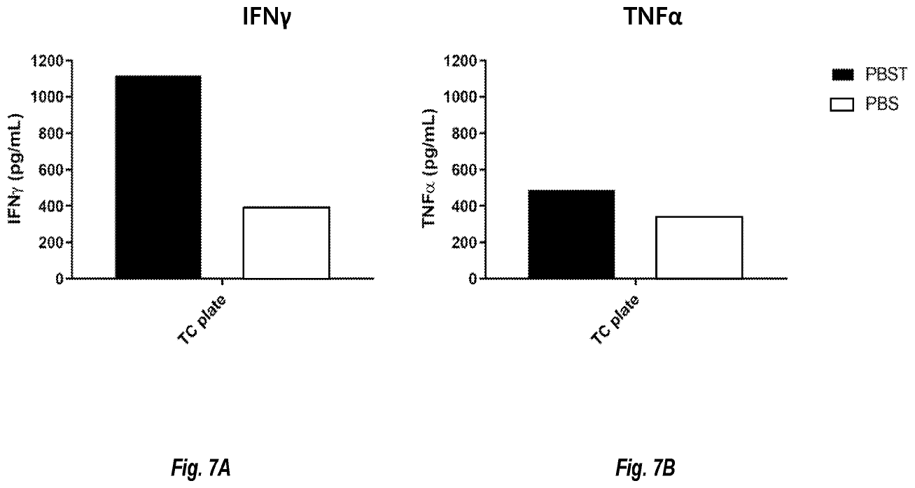

The comPACT coated plate assay was performed with Neo12 TCR T cells as described in Example 1, with wash buffer including Tween20 and with wash buffer excluding Tween20. Detection of IFNγ and TNFα was performed with the CBA, as described in Example 1. The results, shown in FIG. 7, show that inclusion of Tween20 in the wash buffer increased cytokine signal for both IFNγ (FIG. 7A) and TNFα (FIG. 7B).

### Example 12. Comparison of Cell-Based Assay and comPACT Coated Plate Assay

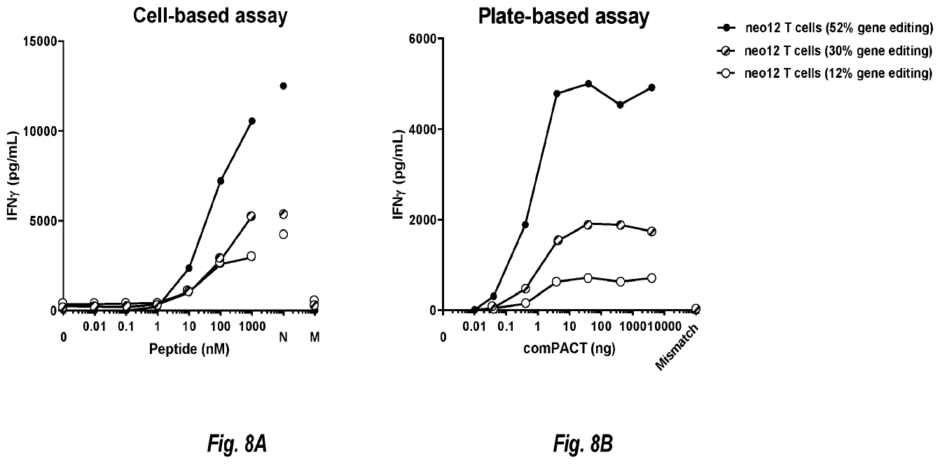

A cell-based assay and the comPACT coated plate assay formats were directly compared using Neo12 TCR T cells expressing Neo12 TCR with different gene editing efficiencies (12%, 30%, or 52%). The comPACT coated plate assay was performed and IFNγ release by the Neo12 TCR T cells was analyzed as described in Example 1. The cell-based assay was performed with the Neo12 TCR T cells co-cultured with K562 cells expressing HLA-A02; or co-cultured with Neo12 HLA-A2 K562 cells (“N” in FIG. 8A) or MART-1 HLA-A2 K562 cells (mismatched cells, “M” in FIG. 8B) that constitutively express the Neo12 or MART1 epitope peptide-HLA complex, respectively. The HLA-A02 expressing K562 cells were pulsed with different concentrations of the same comPACT peptides used in the coated plate assay (at 0-1000 nM) in RPMI medium for 1 hour at 37° C., 5% CO₂. After incubation, the cells were washed twice in RPMI and resuspended in TexMACS supplemented with 3% human AB serum at 250,000 cells/mL. The K562 cells constitutively expressing matched or mismatched epitope peptide-HLA complex were resuspended in TexMACS at 250,000 cells/mL. Neo12 TCR T cells were washed twice with TexMACS and resuspended at 1 million cells/mL. 25,000 peptide-pulsed K562 cells expressing HLA-A02 and K562 cells constitutively expressing epitope peptide-HLA complex were plated in a round bottom microtiter 96-well plate. 100,000 Neo12 TCR T cells were added to each well. The co-culture cell-based assay plate was incubated at 37° C., 5% CO₂, and IFNγ was measured as described in Example 1.

The results, shown in FIG. 8 and the tables below, show that the coated plate assay format (FIG. 8B) concurred with the cell-based assay (FIG. 8A) in measuring the antigen-specific T cell response. Both assays showed a similar trend of IFNγ production, and neither assay showed significant cytokine release in the presence of mismatched epitope peptides. The coated plate assay showed better quantitative correlation between the gene editing efficiency of the Neo12 TCR T cells and cytokine release.

TABLE 8
IFNγ secretion (pg/mL) by T cells in cell-based assay

| | Neo 12 T | Neo 12 T | Neo 12 T |
|---|---|---|---|
| | cells with 10% | cells with 30% | cells with 52% |
| comPACT peptide | gene editing | gene editing | gene editing |
| (nM) | efficiency | efficiency | efficiency |
| 1000 | 2966 | 5260 | 10556 |
| 100 | 2570 | 2786 | 7216 |
| 10 | 1038 | 1008 | 2364 |
| 1 | 423 | 333 | 213 |
| 0.1 | 380 | 195 | 0 |
| 0.01 | 347 | 220 | 0 |
| 0 (no peptide) | 354 | 244 | 0 |
| 0 (“N,” Neo12 | 4204 | 5339 | 12504 |
| HLA-A2 K562 cells) | | | |
| 0 (“M,” MART1 | 436 | 271 | 0 |
| HLA-A2 K562 cells) | | | |

TABLE 9
IFNγ secretion (pg/mL) by T cells in coated plate based assay

| | Neo 12 T | Neo 12 T | Neo 12 T |
|---|---|---|---|
| | cells with 10% | cells with 30% | cells with 52% |
| comPACT peptide | gene editing | gene editing | gene editing |
| (ng/well) | efficiency | efficiency | efficiency |
| 4000 | 708 | 1739 | 4919 |
| 400 | 627 | 1886 | 4537 |
| 40 | 719 | 1910 | 5001 |
| 4 | 628 | 1518 | 4784 |
| 0.4 | 139 | 465 | 1887 |
| 0.04 | 40 | 51 | 306 |
| 0 (no peptide) | 8 | 4 | 0 |
| Mismatched peptide | 0 | 4 | 0 |

1. A method of evaluating a Cell Product, comprising contacting the Cell Product with a polypeptide, wherein the polypeptide comprises an epitope peptide sequence, an HLA class I sequence, and a β2-microglobulin sequence, and forms an epitope peptide-HLA complex; wherein the Cell Product comprises a plurality of T cells, wherein at least one T cell of the Cell Product expresses a select T cell receptor (TCR), wherein the select TCR recognizes the epitope peptide-HLA complex; and detecting at least one marker of T cell function, wherein at least one marker of T cell function is selected from a proliferation marker, a cytotoxicity marker, and an activation marker.

2. The method of claim 1, wherein the polypeptide is bound to a solid support.

3. The method of claim 2, wherein the solid support is a well of a multiwell plate, beads, or cells.

4. The method of claim 2 or claim 3, wherein the polypeptide comprises or is conjugated to a first member of a binding pair, and the solid support comprises a second member of a binding pair.

5. The method of claim 4, wherein the binding pair is biotin and streptavidin or biotin and avidin.

6. The method of any one of claims 2-5, wherein the solid support is a streptavidin-coated multiwell plate.

7. The method of any one of claims 2-5, wherein the solid support is streptavidin-coated beads.

8. The method of claim 7, wherein the beads have an average diameter of 1-10 μm.

9. The method of any one of claims 2-5, wherein the solid support is cells, and wherein the cells have streptavidin-conjugated antibodies bound to the surface.

10. The method of any one of claims 6-9, wherein the polypeptide is conjugated to biotin.

11. The method of any one of claims 1-10, wherein the proliferation marker is selected from Ki67, cell proliferation dyes, Proliferating Cell Nuclear Antigen (PCNA), and Minichromosome Maintenance Complex Component 2 (MCM2).

12. The method of claim 11, wherein Ki67 is detected using an anti-Ki67 antibody.

13. The method of claim 12, wherein the T cells are permeabilized prior to contact with the anti-Ki67 antibody.

14. The method of any one of claims 1-13, wherein the proliferation marker is detected using flow cytometry.

15. The method of any one of claims 1-14, wherein the cytotoxicity marker is selected from CD107a, perforin, and granzyme B.

16. The method of claim 15, wherein CD107a is detected using an anti-CD107a antibody.

17. The method of claim 15 or claim 16, wherein the cytotoxicity marker is detected using flow cytometry.

18. The method of any one of claims 1-17, wherein the activation marker is selected from IFN-γ, IL2, TNFα, 4-1BB, OX40, and CD25.

19. The method of claim 18, wherein the activation marker is detecting using a cytometric bead array.

20. The method of any one of the preceding claims, wherein the Cell Product comprises CD8+ and/or CD4+ T cells from a subject.

21. The method of claim 20, wherein the subject has cancer.

22. The method of any one of the preceding claims, wherein the Cell Product is enriched for T cells that express the select TCR.

23. The method of any one of the preceding claims, wherein the Cell Product has been engineered to express the select TCR.

24. The method of claim 23, wherein the select TCR is preset in the T cell genome at the endogenous TCR locus.

25. The method of claim 23 or claim 24, wherein the Cell Product is produced by a non-viral engineering method.

26. The method of any one of claims 23-25, wherein the Cell Product is produced using CRISPR.

27. The method of any one of claims 23-26, wherein the Cell Product does not comprise any exogenous DNA sequences in the genome of the T cells.

28. The method of any one of the preceding claims, wherein the select TCR binds a neoepitope comprising an amino acid mutation resulting from a somatic coding mutation present in a cancer.

29. The method of claim 28, wherein the Cell Product comprises CD8+ and/or CD4+ T cells from a subject with cancer, and wherein the neoepitope comprises an amino acid mutation resulting from a somatic coding mutation present in the subject's cancer.

30. The method of any one of the preceding claims, wherein the Cell Product is a therapeutic product.

31. The method of any one of the preceding claims, wherein the method comprises determining the percentage of T cells in the Cell Product that express the select TCR.

32. The method of any one of the preceding claims, wherein the method comprises:

33. contacting a solid support with the polypeptide, wherein the polypeptide comprises a first member of a binding pair and the solid support comprises a second member of a binding pair;

34. removing unbound polypeptide;

35. contacting the solid support-bound polypeptide with the Cell Product under conditions suitable for the select TCR to bind to the epitope peptide-HLA complex; and

36. detecting at least one marker of T cell function.

37. The method of any one of the preceding claims, wherein the method comprises determining whether the Cell Product meets a quality control standard.

38. The method of any one of the preceding claims, wherein the method comprises determining the appropriate dose of the Cell Product to administer to a subject.

39. The method of any one of the preceding claims, wherein the method further comprises administering the Cell Product to a subject.

40. The method of claim 34 or claim 35, wherein the subject has cancer.

41. The method of claim 36, wherein the cancer is selected from melanoma, lung cancer, breast cancer, head and neck cancer, ovarian cancer, prostate, and colorectal cancer.

42. The method of any one of the preceding claims, wherein the method comprises determining the affinity of the TCR for the epitope peptide-HLA complex.

43. The method of any one of the preceding claims, wherein the method comprises determining the avidity of the TCR for the epitope peptide-HLA complex.

44. The method of any one of the preceding claims, wherein the method comprises repeating the contacting the Cell Product with the polypeptide until the Cell Product is exhausted.
